(19) United States
(12) Patent Application Publication
(10) Pub. No.: US 2024/0390164 A1
(43) Pub. Date: Nov. 28, 2024

(54) BIOMEDICAL FINGER ASSEMBLY FOR USE WITH CAPACITIVE PANELS AND METHODS FOR MANUFACTURING SAME

(71) Applicant: RCM Enterprise L.L.C., Olympia, WA (US)

(72) Inventors: Erich Theodore Griebling, Issaquah, WA (US); Rachel Adsit Lowing, Leavenworth, WA (US); Ana Rosa Acevedo, Olympia, WA (US)

(21) Appl. No.: 18/674,586

(22) Filed: May 24, 2024

Related U.S. Application Data
(60) Provisional application No. 63/504,423, filed on May 25, 2023.

(51) Int. Cl. A61F 2/58; A61F 2/70

(52) U.S. Cl. CPC A61F 2/586; A61F 2002/701; A61F 2240/001

(57) ABSTRACT

The present technology is generally directed to a prosthetic digit usable with capacitive panels. The digit includes a proximal body removably couplable to a residuum of a user, an intermediate body pivotably coupled to the proximal body via a first fastener, a distal body pivotably coupled to the intermediate body via a second fastener, a first bearing capacitively coupled to the first fastener, and a second bearing capacitively coupled to the second fastener. The digit further includes a first conductive epoxy portion coupled between the first and second bearings, and a second conductive epoxy portion coupled between the second fastener and the distal body. The first and second conductive epoxy portions provide a low impedance, capacitive coupling pathway between the capacitive panel and the residuum of the user.

### CROSS-REFERENCE TO RELATED APPLICATION(S)

The present application claims the benefit of U.S. Provisional Patent Application No. 63/504,423, filed May 25, 2023, the disclosure of which is incorporated herein by reference in its entirety.

### TECHNICAL FIELD

The present technology generally relates to a finger or thumb prosthesis compatible for use with capacitive panels and, more particularly, for use with capacitive touchscreens.

### BACKGROUND

Partial hand loss is the most common upper extremity amputation and has historically been underserved by conventional treatment. Most partial hand amputations are traumatic in origin, and many amputations occur in workplaces where manual labor is performed. Partial hand loss alters the ability to perform important tasks, such as sorting mail, playing an instrument, returning to a vocation, and using electronic devices. Among other difficulties, amputees who wear prosthetic digits can experience limitations in operation of consumer and commercial capacitive panel devices when the prosthesis does not include compatibility with capacitive touchscreens.

Normal operation of a capacitive panel (e.g., the touchscreen of a smartphone) requires a finger or an object to alter the capacitance at the point of contact with the touchscreen. Capacitive touchscreen technology is an industry standard for gaming, signage, and mobile devices such as smartphones. Capacitive touchscreen technology can operate based on mutual or self-capacitance paradigms, which detect touch by sensing the capacitive load of a finger or device when it comes into proximity of the screen. The grid of electrodes of the capacitive touchscreen then sends signals to software to detect finger location.

Upper extremity prosthetic digits can be formed from metallic structural portions and covered with a silicone glove or plastic fairings. The coverings can appear as skin, protect underlying electronics, renew high-wear surfaces by replacement, and improve grip during object handling. Conventional metallic structural prosthetic digits typically use a continuous conductive pathway from the point of contact to the metallic structure (or other component, such as a motor housing) to operate a capacitive panel. Other conventional prosthetic digits can be configured to operate capacitive panels by including a direct conductive pathway from the interfacing prosthetic fingertip to the skin of the user (i.e., using the skin as the capacitive sink). Forming a direct pathway has several limitations, including increased manufacturing complexity and difficulty in maintaining the conductive path through articulating joints.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIGS. 1A and 1B are top and bottom views, respectively, of a prosthetic digit configured in accordance with an embodiment of the present technology.

FIG. 1C is a side view of the prosthetic digit of FIG. 1A.

FIG. 1D is a cross-sectional side view of the prosthetic digit of FIG. 1A, sectioned along the line 1D-1D shown in FIG. 1A.

FIG. 1E is a cross-sectional side view of an intermediate body of the prosthetic digit of FIG. 1A, sectioned along the line 1D-1D shown in FIG. 1A.

FIG. 1F is a cross-sectional top view of a joint of the prosthetic digit of FIG. 1A, sectioned along the line 1F-1F shown in FIG. 1C.

FIG. 2 illustrates a bearing configured in accordance with an embodiment of the present technology.

FIG. 3A is a perspective view of a fascia configured in accordance with an embodiment of the present technology.

FIG. 3B is a cross-sectional side view of the prosthetic digit of FIG. 1A sectioned along the line 1D-1D shown in FIG. 1A, including the fascia of FIG. 3A.

FIGS. 4A and 4B illustrate a user wearing a prosthetic digit configured in accordance with an embodiment of the present technology.

FIG. 5 is a flowchart illustrating a method of manufacturing a prosthetic digit configured in accordance with an embodiment of the present technology.

### DETAILED DESCRIPTION

### A. Overview

The present technology is directed to a prosthetic digit that enables use of capacitive panel control interfaces, such as touchscreens on various consumer and commercial electronic devices (e.g., smartphones, tablets, laptops, printers, machinery, etc.). Prosthetic digits configured in accordance with the present technology, when worn as a prosthetic thumb or finger, are configured to provide usability of an electronic device having a capacitive panel by including one or more series coupled capacitors to alter the capacitance at the point of contact with the panel.

It is desirable for prosthetic digits to be lightweight, compact, strong, and incorporate natural joint movement. In lightweight prostheses, or prostheses with one or more articulation points, adding a direct conductive pathway between a capacitive sink and the prosthetic fingertip can increase complexity and decrease reliability of the conductive pathway. Further, while adding a heavy metallic sink can provide an endpoint for the conductive pathway, the metallic sink can interfere with use of the prosthesis by increasing the weight of the device. Digits configured in accordance with the present technology are expected to restore the ability to operate capacitive panels by providing a lightweight digit having a capacitively coupled pathway to the skin of the user. Given increasingly prolific capacitive panel integration in consumer and commercial electronics, the ability to operate such panels is expected to increase independence in daily activities and restore related portions of vocational efficiency.

The digits disclosed herein for use with a capacitive panel generally include a body, a conductive tip, and a low impedance, series capacitive pathway extending between a residuum of a user and the conductive tip. The pathway generally includes a conductive material (e.g., a carbon nanotube loaded epoxy) disposed in one or more channels formed in the body and other conductive components, such as fasteners and bearings disposed in hinges of the digit. The conductive tip may extend the series capacitive pathway to the electrodes of the capacitive panel to aid in capacitive coupling between the touchscreen and the conductive material. The conductive tip pad may be configured to interact with the capacitive panel similarly to a user's intact fingertip.

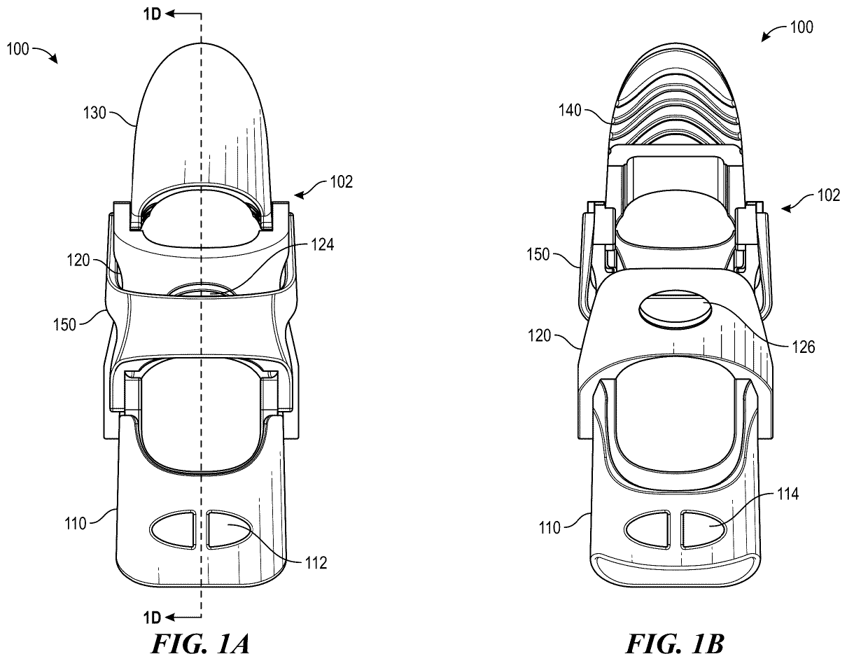

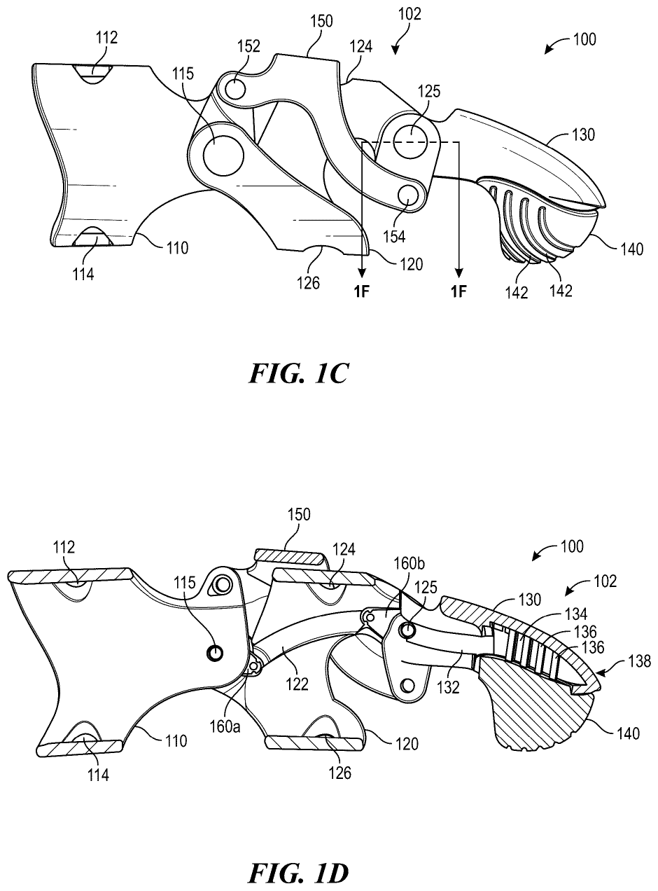

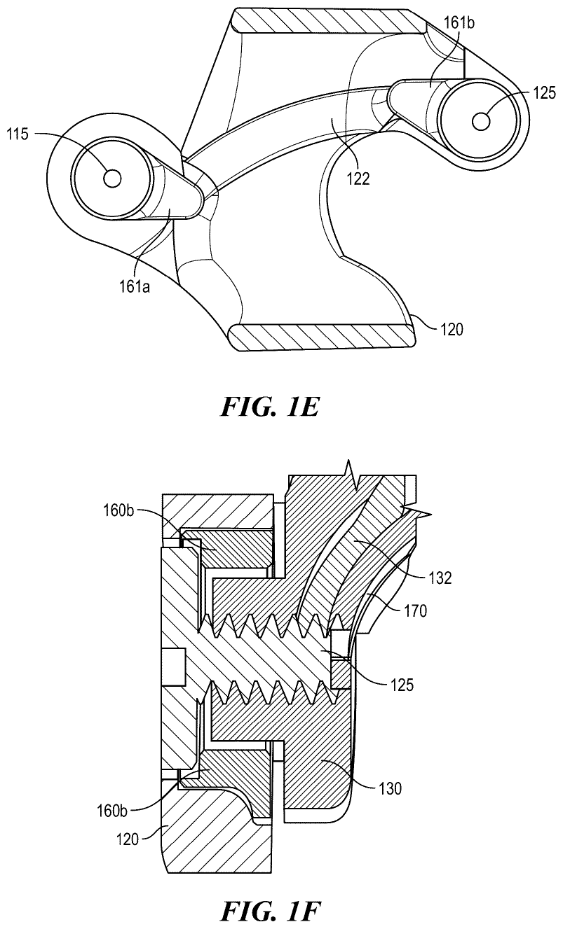

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims, but are not described in detail with respect to FIGS. 1-5.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

### B. Selected Embodiments of Prosthetic Digits Compatible for Use with Capacitive Panels

FIGS. 1A and 1B are top and bottom views, respectively, of a prosthetic digit 100 (“digit 100”) configured in accordance with an embodiment of the present technology. The digit 100 is configured to allow a user to control a capacitive panel (not shown) via contact of the digit 100 to the panel through low impedance, series capacitive coupling. The digit 100 includes a body 102 having a distal body portion 130 (“a first body portion”), an intermediate body portion 120 (“a second body portion”), a proximal body portion 110 (“a third body portion”), and a tendon body portion 150 (“a fourth body portion”). The proximal body portion 110, the intermediate body portion 120, and/or the distal body portion 130 can each be sized and shaped (e.g., cylindrically shaped) for receiving a residuum of a user. The digit 100 also includes a conductive tip 140 (FIG. 1B) coupled to a bottom side of the distal body portion 130. The proximal body portion 110, the intermediate body portion 120, the distal body portion 130, and the tendon body portion 150 can each be formed from a suitable lightweight material, such as plastic, carbon fiber, titanium, nylon, etc. In some embodiments, one or more of the proximal body portion 110, the intermediate body portion 120, the distal body portion 130, and the tendon body portion 150 can be vapor smoothed (which seals the surface and is expected to eliminate the need for additional exterior coatings), and subsequently dyed. In some embodiments, the proximal body portion 110, the intermediate body portion 120, the distal body portion 130, and/or the tendon body portion 150 can be fabricated via additive manufacturing (e.g., 3D printing techniques) or other suitable manufacturing processes.

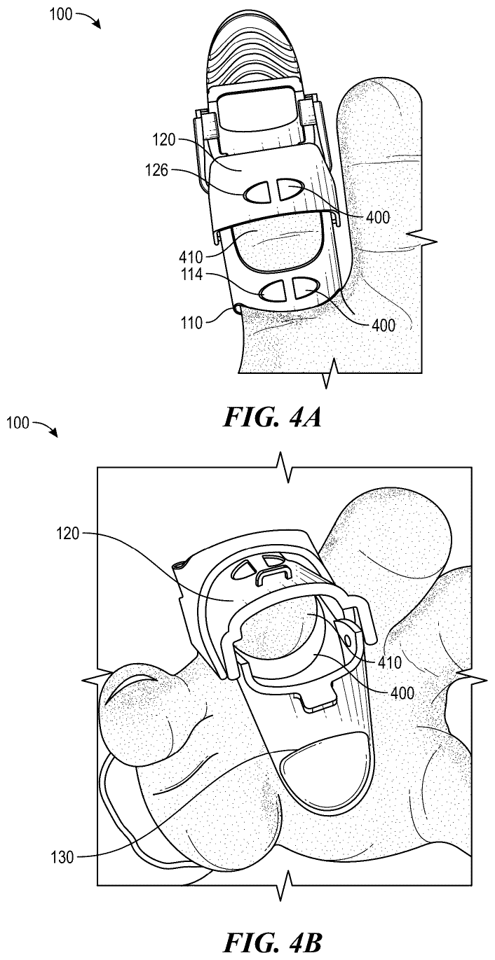

The proximal body portion 110 can include one or more proximal dorsal shim holes 112 and one or more proximal palmar shim holes 114. The intermediate body portion 120 can include one or more intermediate dorsal shim holes 124 and one or more intermediate palmar shim holes 126. The digit 100 may include a mounting feature (e.g., a socket (partial, radial, etc.), frame, strap, shim, or any other suitable mounting feature) for suspending the digit 100 from a residuum of a user. In some embodiments, the mounting feature may be formed from a biocompatible material. One embodiment of the shim is shown in FIGS. 4A and 4B. The shim can be coupled to any one or more of the proximal dorsal shim holes 112, the proximal palmar shim holes 114, the intermediate dorsal shim holes 124, and the intermediate palmar shim holes 126. The shim can be configured to help secure the digit 100 to the remaining residuum in the approximate position of anatomic digits of the user. In some embodiments, it is possible to mount the digit 100 in a fashion that is non-anatomic, such as in the case of unique clinical presentations.

FIG. 1C is a side view of the digit 100. As best seen in FIG. 1C, the intermediate body portion 120 is pivotably coupled to the proximal body portion 110 via first fasteners 115 (one in view, another on the other side of the digit 100). The distal body portion 130 is pivotably coupled to the intermediate body portion 120 via second fasteners 125. The tendon body portion 150 is pivotably coupled to the proximal body portion 110 and the distal body portion 130 via first and second tendon fasteners 152 and 154, respectively. Each of the fasteners 115, 125, 152, and 154 can be a screw, a bolt, a threaded rod, a pin, etc. The fasteners 115, 125, 152, and 154 define hinges that operate together to approximate the natural movement of a finger. In some embodiments, the digit 100 includes fewer or more body portions than in the illustrated embodiment. For example, the digit 100 may not include the intermediate body portion 120, and the proximal body portion 110 may be pivotably coupled to the distal body portion 130 instead.

As mentioned previously, the conductive tip 140 is positioned and configured to interface with a capacitive panel (not shown). The conductive tip 140 may be operably coupled to the distal body portion 130 with a suitable attachment feature, such as interference fit, fasteners, non-conductive adhesive, etc. The conductive tip 140 further comprises a plurality of grip indentations 142 formed on an interfacing surface of the conductive tip 140. Such features are expected to improve object handling for the user of the digit 100. In other embodiments, the grip indentations 142 may have a different arrangement/pattern. The conductive tip 140 may be formed from a conductive material, such as conductive thermoplastic polyurethane (TPU), conductive silicone (e.g., silicone having conductive filler, carbon nanotube-loaded silicone), conductive elastomer (e.g., thermoplastic elastomer (TPE)), polymer, film, paper, fabric, metal, or other suitable conductive materials.

FIG. 1D is a cross-sectional side view of the digit 100, sectioned along the line 1D-1D shown in FIG. 1A. As best seen in FIG. 1D, the joints between the proximal body portion 110 and the intermediate body portion 120 are formed by the first fasteners 115 and first bearings 160a. Similarly, the joints between the intermediate body portion 120 and the distal body portion 130 are formed by the second fasteners 125 and second bearings 160b. The first and second bearings 160a and 160b are described in further detail below.

The intermediate body portion 120 includes a first channel 122 and the distal body portion 130 includes a second channel 132. The first and second channels 122 and 132 are sized and shaped to be at least partially filled with a material that defines a low impedance conductive pathway along the digit 100. In the illustrated embodiment, for example, the first and second channels 122 and 132 are at least partially filled with conductive material. The conductive material can serve as a low impedance conductive path that forms part of a series capacitive pathway, which will be described in further detail below. The first and second channels 122 and 132 may be recessed channel regions formed together with the corresponding body portions 120 and 130 (e.g., via additive manufacturing). In other embodiments, the first and second channels 122 and 132 can be formed by removing/carving out material from the corresponding body portions 120 and 130. In still other embodiments, other suitable techniques may be used to form the first channel 122 and/or second channel 132. The distal body portion 130 includes a cavity 134 and a plurality of struts 136 in a distal end portion 138 of the digit 100. In some embodiments, the cavity 134 is also at least partially filled with the conductive material (or other material that can define a low impedance conductive pathway along the digit 100) and is connected (physically and/or electrically) to the second channel 132.

In some embodiments, the conductive material may comprise conductive epoxy. For example, the conductive epoxy may comprise carbon nanotube (CNT)-loaded epoxy. The CNT-loaded epoxy can have a CNT loading ratio or concentration of between approximately 0.5% and 2% by weight (e.g., 0.7%, 1%, 1.5%). In other embodiments, however, the CNT-loaded epoxy can have any CNT loading ratio or concentration depending on specific needs. For example, a high CNT concentration may increase conductivity, but may also make the epoxy less flexible and increase overall manufacturing costs. In some embodiments, MED-301-2FL epoxy, which is biocompatible, low-temperature curing, and flexible, is used. In still other embodiments, other suitable materials may be used within the first channel 122, the second channel 132, and/or the cavity 134.

In some embodiments, one or both of the first and second channels 122 and 132 and the conductive material disposed therein are replaced by other conductive pathway-forming structures or conductive material portions including, but not limited to, wires, braided cables, malleable resin or metal, metal-plated custom inserts (e.g., additive plastic plated with silver or other metal), direct-printed custom metal inserts, machined custom inserts, carbon fiber, powdered metallurgy inserts (e.g., manufactured via metal injection molding (MIM)), etc.

FIG. 1E is a cross-sectional side view of the intermediate body 120 of the digit 100, sectioned along the line 1D-1D shown in FIG. 1A. FIG. 1F is a cross-sectional top view of the joint between the intermediate body portion 120 and the distal body portion 130, sectioned along the line 1F-1F shown in FIG. 1C. For purposes of clarity and illustration, the other portions of the digit 100 are not illustrated. Referring to FIGS. 1E and 1F together, the first and second bearings 160a and 160b are installed onto first and second bearing receiving portions 161a and 161b of the intermediate body portion 120 while the second fastener 125 is coupled to (e.g., threaded onto) the distal body portion 130. Similarly, while not shown, the first fastener 115 can be coupled to the proximal body portion 110. The first and second fasteners 115 and 125, and the first and second bearings 160a and 160b form a series capacitive pathway from the residuum of the user (e.g., disposed in the proximal body portion 110) and the conductive tip 140.

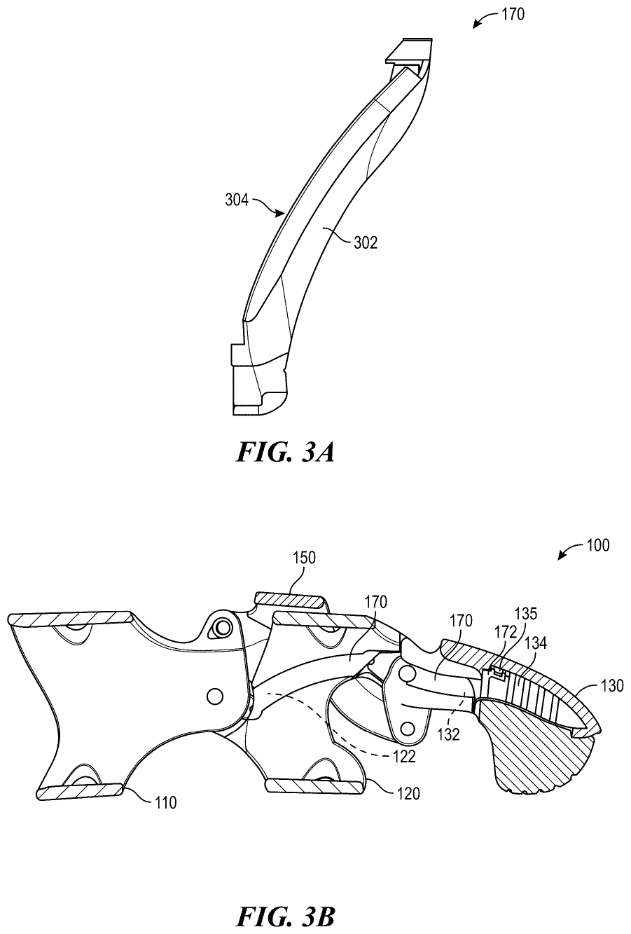

As best seen in FIG. 1E, once the conductive material is disposed to at least partially fill the first channel 122, the conductive material (e.g., conductive epoxy) may cure while contacting both the first bearing 160a and the second bearing 160b, thereby forming a direct electrical pathway between the first and second bearings 160a and 160b. Referring to FIG. 1F, once the conductive material is disposed to at least partially fill the second channel 132, the conductive material may cure while contacting the second fastener 125, thereby forming a direct electrical pathway between the second fastener 125 and the conductive material in the cavity 134. In some embodiments, a fascia 170 can be applied to the conductive material in the first and/or second channels 122 and 132, as will be described in greater detail below with respect to FIGS. 3A and 3B.

With continued reference to FIG. 1F, the second fastener 125 and the second bearing 160b are not in direct physical contact but are instead capacitively coupled to one another with the distal body portion 130 acting as a dielectric. In this way, the conductive material in the cavity 134 is capacitively coupled to the second bearing 160b. While not shown, the first fastener 115 and the first bearing 160a may be arranged in a similar manner to the arrangement shown in FIG. 1F such that the first fastener 115 and the first bearing 160a are capacitively coupled to one another.

Referring to FIGS. 1C, 1D, and 1F together, as a result of the disclosed configuration of the digit 100, once the residuum of the user (not shown) is disposed in the proximal body portion 110 and/or the intermediate body 120, and proximate to the first fastener 115 and/or the conductive material (with or without a conductive shim), a series capacitive pathway is formed to the conductive material in the cavity 134. The series capacitive pathway can include a first capacitive coupling between the first fastener 115 and the first bearing 160a, a first direct coupling between the first and second bearings 160a and 160b, a second capacitive coupling between the second bearing 160b and the second fastener 125, and a second direct coupling between the second fastener 125 and the conductive material in the cavity 134.

Referring again to FIG. 1D, the conductive tip 140 is configured to capacitively bridge the distance between a capacitive panel (not shown) and the conductive material filled in the cavity 134 to extend the series capacitive pathway/coupling. Capacitive coupling (e.g., electric field or electrostatic coupling) does not require contact between the capacitive panel and the capacitive body (e.g., the residuum of the user) for use of the capacitive panel. In this regard, the conductive tip 140 is not in direct electrical communication with the conductive material in the cavity 134. As is known to those of skill in the art, capacitance between the two objects is a function of the surface area of the objects, the distance between the objects, and the permittivity (i.e., the ability of a substance to store electrical energy in an electric field). A threshold capacitance value can mimic the human body to operate the capacitive panel. In some embodiments, the series capacitive pathway from the first fastener (or a conductive shim) to the conductive tip 140 provides a circuit of sufficiently low impedance that electrically couples the conductive tip 140 to a capacitive body (i.e., virtual ground), and provides a threshold capacitance value to operate the capacitive panel. In some embodiments, the elements of the series capacitive pathway can serve as the capacitive body if the elements are far enough away from the capacitive panel (e.g., in a relatively large digit 100). In some embodiments, the residuum of the user can serve as the capacitive body (e.g., in a relatively small digit 100). In some embodiments, the digit 100 may be used with the capacitive panel without the conductive tip 140, such as in embodiments where the distance between the conductive material in the cavity 134 and the capacitive panel is relatively short. Other configurations are also within the scope of the present disclosure.

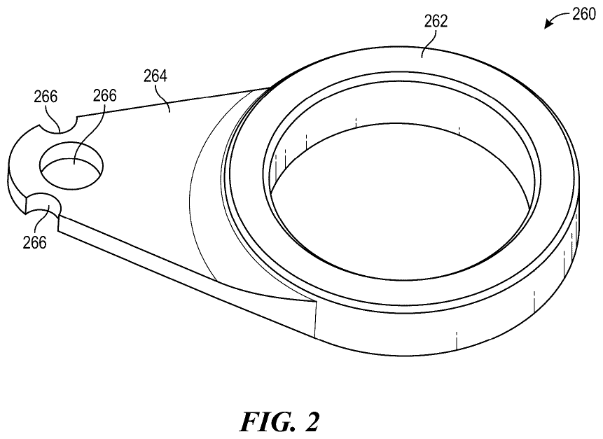

FIG. 2 is a perspective view of a bearing 260 configured in accordance with an embodiment of the present technology. In some embodiments, the bearing 260 illustrated in FIG. 2 is identical to the first and second bearings 160a and 160b illustrated in FIGS. 1D and 1E. In other embodiments, however, the first and second bearings 160a and 160b are different from one another and/or different from the bearing 260 illustrated in FIG. 2. Further, while only a single bearing 260 is illustrated in FIG. 2, it will be appreciated that additional bearings to be installed with the digit 100 (FIGS. 1A-1D) or other suitable digits will have identical or very similar configurations.

The bearing 260 has a circular washer portion 262 configured to capacitively couple to a fastener (e.g., fastener 115, fastener 125) and provide conventional bearing functionality, as shown and described above with reference to FIG. 1F. The bearing 260 also includes an extruded portion 264 sized and shaped such that when the bearing 260 is installed with a fastener (e.g., fastener 115, fastener 125), the extruded portion 264 is at least partially positioned in the first channel 122 and the conductive material (e.g., conductive epoxy) disposed in the first channel 122 can cure on or around the extruded portion 264 for better attachment. In some embodiments, the extruded portion 264 may further include one or more cutouts 266 that provide increased surface area onto which the conductive material (e.g., conductive epoxy) can contact and cure. In further embodiments, the cutouts 266 may have a different size/arrangement relative to each other and/or the extruded portion 264. Further, the cutouts 266 are an optional feature that may not be included in some embodiments.

In some embodiments, the circular washer portion 262 of the bearing 260 can have a diameter between 3 mm and 7 mm (e.g., 5 mm) and the extruded portion 264 can extend from the washer portion 262 by a length of between 1 mm and 3 mm (e.g., 2 mm) such that the bearing 160 may have an overall dimension of around 5 mm by 7 mm. In some embodiments, the bearing may have a thickness less than 5 mm (e.g., 1 mm, 2 mm, 3 mm, 4 mm). In other embodiments, however, the dimensions of the bearing 260 may vary depending upon the size of the corresponding fastener(s) and/or digit onto which the bearing 260 will be installed. The bearing 260 can be composed of metal (e.g., stainless steel) or other suitable materials.

FIG. 3A is a perspective view of a fascia component 170 configured in accordance with an embodiment of the present technology. In some embodiments, the digit 100 (FIGS. 1A-1D) can include one or more fascia components 170 to cover the conductive material disposed in the first and second channels 122 and 132 and/or the cavity 134. The fascia component 170 includes a first side 302 and an opposing second side 304. The first side 302 of the fascia component 170 can provide an improved aesthetic appearance to the digit 100, such as by being a different color from the conductive material and/or providing a different texture placed on the conductive material. In some instances, including the fascia component 170 to cover and protect the conductive material within the channels is expected to be more efficient and reliable than coating portions of or the entire digit 100. The second side 304 of the fascia component 170 is configured to be attached to the conductive material directly (e.g., in contact with conductive epoxy while the epoxy is curing) or indirectly (e.g., via an adhesive). The fascia component 170 can be fabricated via additive manufacturing (e.g., 3D printing techniques) or other suitable manufacturing processes.

FIG. 3B is a cross-sectional side view of the prosthetic digit 100 sectioned along the line 1D-1D shown in FIG. 1A with installed fascia components 170. In the illustrated embodiment, for example, fascia components 170 are applied to cover the conductive material in both the first channel 122 and the second channel 132. In particular, the fascia component 170 covering the second channel 132 can also at least partially cover the cavity 134. In some embodiments, for example, the fascia component 170 covering the second channel 132 can include an attachment feature 172 configured to releasably couple to a protrusion 135 in the cavity 134 to further secure the fascia 170 in place. In some embodiments, the fascia component 170 may be composed of a conductive material. The fascia component 170 may be coated, plated, and/or otherwise modified with conductive material. In other embodiments, the digit 100 may not include the conductive material and instead have the fascia component 170 configured to form the series capacitive pathway. As noted above, in still further embodiments the fascia components 170 may have other configurations/arrangements. Further, the fascia components 170 are optional features that may not be included in some embodiments.

FIGS. 4A and 4B illustrate a user wearing the digit 100 configured in accordance with embodiments of the present technology. Referring to FIGS. 4A and 4B together, a residuum 410 of the user is disposed in the proximal body portion 110 and secured by two shims 400. The shims 400 are sized and shaped to secure the digit 100 to the residuum 410 in the approximate position of anatomic digits of the user. As discussed above, the shims 400 can be coupled (e.g., via snapping, interference fit) to any one or more of the proximal dorsal shim holes 112 (not shown), the proximal palmar shim holes 114, the intermediate dorsal shim holes 124 (not shown), and the intermediate palmar shim holes 126. The user may switch between shims of varying sizes to, for example, adjust for changes in the volume of the residuum 410. The shims 400 can be made from a conductive material (e.g., CNT-loaded silicone) or any other suitable material (e.g., a biocompatible material). In some embodiments, the shims 400 form part of the series capacitive pathway by electrically coupling the residuum 410 to the first fastener 115, the second fastener 125, and/or the conductive material. In other embodiments, however, the user may wear the digit 100 without using any shims 400.

### C. Method of Manufacturing Prosthetic Digits Compatible for Use with Capacitive Panels

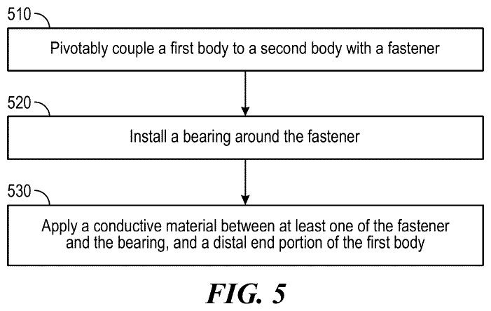

FIG. 5 is a flowchart 500 illustrating a method 500 of manufacturing a prosthetic digit configured in accordance with an embodiment of the present technology. The method 500 begins at block 510 with pivotably coupling a first body to a second body with a fastener. At block 520, the method 500 includes installing a bearing around the fastener. At block 530, the method 500 includes disposing or applying a conductive material between at least one of the fastener and the bearing, and a distal end portion of the first body. In some embodiments, for example, the conductive material can be disposed via a pressure-assisted syringe dispenser. In other embodiments, however, other suitable techniques may be used to dispose the conductive material. In some embodiments, the first body includes a channel extending between at least one of the fastener and the bearing, and the distal end portion of the first body, and the method includes disposing or applying the conductive material in the channel.

In some embodiments, the conductive material comprises conductive epoxy and the method 500 can further include curing the conductive epoxy. In one embodiment, for example, the conductive epoxy is cured at a temperature between 70 degrees Celsius and 90 degrees Celsius (e.g., 80 degrees Celsius) for a period of 1 hour or more. In other embodiments, however, other suitable curing parameters may be used.

In some embodiments, the conductive material may comprise CNT-loaded epoxy and the method 500 may further include creating the CNT-loaded epoxy, such as loading CNTs in an epoxy resin at a first concentration (e.g., 1.5% by weight), loading CNTs in an epoxy hardener at a second concentration, and mixing the resin and hardener to form CNT-loaded epoxy with a final concentration (e.g., between 0.5% and 2% by weight, 0.7%).

In additional embodiments, the bearing may include an extruded portion and the method 500 may further include applying the conductive material onto the extruded portion of the bearing. In some embodiments, the method 500 may further include applying the conductive material in a cavity located in a distal end portion of the distal body. In still further embodiments, the method 500 may further include applying fascia components to the exposed surfaces of the conductive material.

In further embodiments, the fastener can be a first fastener, the bearing can be a first bearing, and the method can further include pivotably coupling a third body to the second body with a second fastener, installing a second bearing around the second fastener, and applying the conductive material between the first bearing and the second bearing. In some embodiments, the channel is a first channel and the second body includes a second channel, and the method includes applying the conductive material in the second channel.

### D. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while the channels for conductive material are shown in the embodiment of FIGS. 1D-IF on both the intermediate body and the distal body, in other embodiments the channels may be disposed on only one of the intermediate body or distal body, or also on the proximal body. While steps are presented in a given order, alternative embodiments may perform steps in a different order. Moreover, the various embodiments described herein may also be combined to provide further embodiments. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment.

For ease of reference, identical reference numbers are used to identify similar or analogous components or features throughout this disclosure, but the use of the same reference number does not imply that the features should be construed to be identical. Indeed, in many examples described herein, identically numbered features have a plurality of embodiments that are distinct in structure and/or function from each other. Furthermore, the same shading may be used to indicate materials in cross section that can be compositionally similar, but the use of the same shading does not imply that the materials should be construed to be identical unless specifically noted herein.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms may also include the plural or singular term, respectively. Additionally, the term “comprising” is used throughout to mean including at least the recited feature(s) such that any greater number of the same feature and/or additional types of other features are not precluded. Directional terms, such as “upper,” “lower,” “front,” “back,” “vertical,” and “horizontal,” may be used herein to express and clarify the relationship between various elements. It should be understood that such terms do not denote absolute orientation. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A prosthetic digit for use with a capacitive panel, the prosthetic digit comprising:
a proximal body removably couplable to a residuum of a user;
a distal body pivotably coupled to the proximal body via fastener;
a conductive tip carried by the distal body;
a bearing capacitively coupled to the fastener; and
a conductive material portion electrically coupling the fastener and the conductive tip,
wherein, when the distal body is in proximity to the capacitive panel, a series capacitive pathway between the residuum of the user and the distal body alters the capacitance of the capacitive panel.

2. The prosthetic digit of claim 1 wherein:
the distal body comprises a channel; and
the conductive material portion is disposed in the channel.

3. The prosthetic digit of claim 1 wherein:
the bearing comprises (a) a washer portion capacitively coupled to the fastener, and (b) an extruded portion in contact with the conductive material portion.

4. The prosthetic digit of claim 1, further comprising a shim capacitively coupled to at least one of the bearing, the fastener, and the conductive material portion, wherein the shim is removably couplable to the proximal body, and further wherein the shim is sized and shaped to releasably engage the residuum of the user.

5. The prosthetic digit of claim 1 wherein the conductive material portion is composed of carbon nanotube (CNT)-loaded epoxy.

6. The prosthetic digit of claim 5 wherein the CNT-loaded epoxy has a CNT loading of between 0.5% and 2% by weight.

7. A prosthetic digit for use with a capacitive panel, the prosthetic digit comprising:
a body removably couplable to a residuum of a user, the body comprising:
a proximal body portion removably couplable to the residuum of the user;
an intermediate body portion pivotably coupled to the proximal body portion; and
a distal body portion pivotably coupled to the intermediate body portion;
a conductive tip removably couplable to the distal body portion, wherein the conductive tip is configured to interface with the capacitive panel during use; and
a series capacitive pathway extending between the residuum of the user and the conductive tip, the pathway comprising:
a first fastener pivotably coupling the intermediate body portion to the proximal body portion;
a first bearing capacitively coupled to the first fastener and the residuum of the user;
a second fastener pivotably coupling the distal body portion to the intermediate body portion;
a second bearing capacitively coupled to the second fastener;
a first conductive material portion electrically coupling the first and second bearings;
a second conductive material portion electrically coupling the second fastener and a distal end portion of the distal body portion,
wherein, when the conductive tip is in proximity to the capacitive panel, the series capacitive pathway alters the capacitance of the capacitive panel.

8. The prosthetic digit of claim 7 wherein:
the intermediate body portion comprises a first channel;
the first conductive material portion is disposed in the first channel;
the distal body portion comprises a second channel; and
the second conductive material portion is disposed in the second channel.

9. The prosthetic digit of claim 7 wherein the distal body portion includes a cavity at a distal end portion of the distal body portion, and wherein the second conductive material portion is further disposed in the cavity.

10. The prosthetic digit of claim 9 wherein the distal body portion further includes a plurality of struts positioned within the cavity.

11. The prosthetic digit of claim 7 wherein:
the first bearing comprises (a) a washer portion capacitively coupled to the first fastener, and (b) an extruded portion engaged with the first conductive material portion; and
the second bearing comprises (a) a washer portion capacitively coupled to the second fastener, and (b) an extruded portion engaged with the second conductive material portion.

12. The prosthetic digit of claim 11 wherein:
the extruded portion of the first bearing includes one or more cutouts configured to provide a greater surface area for engagement with the first conductive material portion; and
the extruded portion of the second bearing includes one or more cutouts configured to provide a greater surface area for engagement with the second conductive material portion.

13. The prosthetic digit of claim 7, further comprising:
a first fascia component coupled to and covering the first conductive material portion; and
a second fascia component coupled to and covering the second conductive material portion.

14. The prosthetic digit of claim 7 wherein the series capacitive pathway further comprises a shim capacitively coupled to the first bearing, and wherein the shim is removably couplable to the proximal body portion or the intermediate body potion, and further wherein the shim is sized and shaped to releasably engage the residuum of the user.

15. The prosthetic digit of claim 7 wherein each of the first and second conductive material portions is composed of carbon nanotube (CNT)-loaded epoxy.

16. The prosthetic digit of claim 15 wherein the CNT-loaded epoxy has a CNT loading of between 0.5% and 1% by weight.

17. The prosthetic digit of claim 7 wherein the conductive tip comprises carbon nanotube (CNT)-loaded silicone.

18. The prosthetic digit of claim 7 wherein the body is composed of nylon.

19. A method of manufacturing a prosthetic digit for use with a capacitive panel, the method comprising:
pivotably coupling a first body to a second body with a fastener;
installing a bearing around the fastener; and
applying a conductive material between at least one of the fastener and the bearing, and a distal end portion of the first body.

20. The method of claim 19, further comprising:
curing the conductive material at a temperature between 70 degrees Celsius and 90 degrees Celsius for a period of 1 hour or more.

21. The method of claim 19 wherein the conductive material comprises carbon nanotube (CNT)-loaded epoxy.

22. The method of claim 21, wherein the CNT-loaded epoxy has a CNT loading of between 0.5% and 2% by weight.

23. The method of claim 19, wherein the bearing includes an extruded portion, the method further comprising:
applying the conductive material onto the extruded portion of the bearing.

24. The method of claim 19, wherein the first body includes a cavity at the distal end portion of the first body, the method further comprising:
applying the conductive material in the cavity.

25. The method of claim 19, wherein the fastener is a first fastener, wherein the bearing is a first bearing, the method further comprising:
pivotably coupling a third body to the second body with a second fastener;
installing a second bearing around the second fastener; and
applying the conductive material between the first bearing and the second bearing.
